(19) United States
(12) Patent Application Publication
(10) Pub. No.: US 2003/0144712 A1
(43) Pub. Date: Jul. 31, 2003

(54) Methods for overcoming organ transplant rejection

(76) Inventor: Jackson Streeter, M.D., Reno, NV (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/327,605

(22) Filed: Dec. 20, 2002

Related U.S. Application Data
(63) Continuation-in-part of application No. 10/287,432, filed on Nov. 1, 2002.
(60) Provisional application No. 60/343,399, filed on Dec. 20, 2001.
(60) Provisional application No. 60/354,009, filed on Jan. 31, 2002.
(60) Provisional application No. 60/369,260, filed on Apr. 2, 2002.

(51) Int. Cl. A61N 5/06

(52) U.S. Cl. 607/88; 128/898

(57) ABSTRACT

Therapeutic methods for preventing or retarding organ transplant rejection are described, the methods including delivering to a transplanted organ a rejection effective amount of light energy, the light energy having a wavelength in the visible to near-infrared wavelength range, wherein delivering the rejection effective amount of light energy includes selecting a power density (mW/cm²) of light energy to be received at the organ. The power density is at least about 0.01 mW/cm² and no more than about 100 mW/cm², to be delivered to the transplanted organ after completion of the transplant procedure.

### RELATED APPLICATION INFORMATION

This application claims priority under 35 U.S.C. §119(e) to U.S. Provisional Application Serial No. 60/343,399 filed Dec. 20, 2001, No. 60/354,009 filed Jan. 31, 2002, and No. 60/369,260 filed Apr. 2, 2002. This application is also a continuation-in-part of U.S. patent application Ser. No. 10/287,432, filed Nov. 1, 2002.

### BACKGROUND OF THE INVENTION

1. Field of the Invention

The present invention relates in general to medical procedures for solid organ transplantation, and more particularly to methods for overcoming organ and tissue transplant rejection.

2. Description of the Related Art

Over the past thirty years, solid organ transplantation has become an increasingly viable treatment option for a variety of diseases and conditions. For example, in the United States alone, kidney transplants are now performed at an annual rate of over 9,000, and heart transplants are performed at the rate of over 1,500 per year. However, “true” rejection of the transplanted tissue due to the recipient's normal immune response, and rejection or ultimate loss of the graft due to transplant-related pathophysiology in the graft tissue continue to be major hurdles to successful transplantation. Yet the multiple processes and interactions underlying rejection and ultimate graft loss due transplant-related tissue pathologies remain incompletely understood.

Both specific and non-specific mechanisms are involved in graft rejection. In humans, the immune response underlying true rejection includes two main mechanisms that are characterized by a high level of specificity against antigenic epitopes that are expressed on agents foreign to the body, including various pathogens and transplant cells. The first mechanism is cell-mediated through the T-cell immune response, which triggers production of a variety of cytokines, such as interleukin-1 (IL-1) and tumor necrosis factor (TNFα,β), from various cell populations. Cytokines, particularly IL-1 and TNF, appear to be significantly involved in generating inflammatory responses. The second mechanism is humoral, mediated by antibodies ultimately produced after B cell activation. Nonspecific mechanisms, such as the activation of complement and its soluble factors having vasoactive and chemotactic properties, are also involved in graft rejection and ultimate graft loss.

Rejection is generally clinically classified into three main types that are each characterized by a distinct time course. Hyperacute rejection is characterized by rapid onset of tissue damage within hours or even minutes of transplantation. Acute rejection occurs within days to weeks after transplantation. Chronic rejection is a slower, ongoing process which may begin as early as shortly after transplantation and continue for months or years, ultimately resulting in loss of the graft.

Hyperacute rejection results from activation of complement. While the mechanisms involved in activation of complement are not completely understood, complement activation can be triggered by preexisting antibodies that react specifically with the “foreign” markers expressed by cells in the graft, or from an alternative pathway activated by a variety of nonspecific stimuli related directly or indirectly to organ storage or to the graft procedure itself. Such stimuli include, for example, physical trauma resulting from the surgical procedure, infection, warm ischemia and reperfusion injury, or thrombosis of vessels in the graft. Activation of complement can result in significant tissue damage. Complement is known to be involved in reperfusion injury because complement inhibitors, such as cobra venom factor, decrease reperfusion injury following warm ischemia. Complement activation is known to be involved in rejection of transplanted organ grafts, and is thought to produce much of the tissue injury in transplantation by playing an important role in generating inflammation. In particular, complement factor C5a is involved in recruiting inflammatory cells to sites of injury.

Acute rejection occurs days to weeks after transplantation, and is caused by sensitization of the host to the foreign tissue that makes up the graft. Once the host's immune system has identified the transplanted tissue as foreign, all immune system resources including both specific (antibody and T cell-dependent) responses .and non-specific (phagocytic, complement-dependent, etc.) responses are deployed against the graft.

The processes underlying chronic rejection, which affects a high percentage of kidney and heart allograft recipients in particular, remain poorly understood but likely include several distinct, if not necessarily independent, processes. Chronic rejection can occur due to the long-term administration of immunosuppressive agents to prevent acute rejection. While immunosuppressive therapy may prevent acute rejection, it may also cause the graft tissue to undergo hyperplastic and hypertrophic changes that adversely affect ultimate graft survival. Chronic rejection can also involve accelerated graft atherosclerosis due to vascular endothelial cell damage, which also adversely affects ultimate survival of the graft. In addition, chronic pathologies in the graft tissue that are related to original donor tissue quality and changed workload imposed on the graft tissue result in substantial rates of graft loss.

Nevertheless, the increased availability of effective immunosuppressive agents over the last few decades has contributed greatly to increase the success rate of allograft procedures. These immunosuppressive agents prevent rejection of donor tissue by the recipient's normal immune response. Such agents include, for example, nonspecific cytotoxic agents such as azathioprine and cyclophosphamide, and corticosteroids such as prednisone. More recently, the immunosuppressive agents cyclosporine, tacrolimus and mycophenolate mofetil have become available.

Yet the use of immunosuppressive agents carries several significant limitations. Nonspecific cytotoxic agents work their immunosuppressive effect by targeting rapidly proliferating lymphocytes. Unfortunately, the cytotoxic effect is not limited to lymphocytes but extends to other rapidly proliferating cells, including bone marrow and gastrointestinal cells, thus risking bone marrow suppression and infection. When corticosteroids are used in combination with nonspecific cytotoxic agents, the risk of infection increases still further. The adverse side effects of nonspecific cytotoxic agents can be avoided by using the newer generation agents such as cyclosporine. However, all immunosuppressive agents, by virtue of suppressing the body's normal immune response, increase the risk of infection by pathogens of all types, including bacterial, viral, fungal and other more unusual pathogens. In addition, administration of immunosuppressive agents increases the risk of lymphomas and related malignancies, possibly due to the impaired immune response to viral pathogens. Still further, immunosuppressive agents should have little therapeutic value in preventing graft tissue damage that is not directly related to the immune response.

Thus, methods for overcoming transplant rejection have focused primarily on the processes underlying acute rejection, and have relied mainly on suppressing the normal immune response. Attempts have been made to overcome hyperacute rejection by disrupting the normal function of complement, but have met with limited success. Hyperacute rejection is currently primarily avoided by close tissue typing of organ donors and recipients for histocompatibility. Chronic rejection continues to be incompletely understood and no single therapeutic approach has proven particularly successful.

In the field of surgery, high energy laser radiation is now well accepted as a surgical tool for cutting, cauterizing, and ablating biological tissue. High-energy lasers are now routinely used for vaporizing superficial skin lesions and, and to make deep cuts. For a laser to be suitable for use as a surgical laser, it must provide laser energy at a power sufficient to heat tissue to temperatures over 50° C. Power outputs for surgical lasers vary from 1-5 W for vaporizing superficial tissue, to about 100 W for deep cutting.

In contrast, low level laser therapy involves therapeutic administration of laser energy to a patient at vastly lower power outputs than those used in high energy laser applications, resulting in desirable biostimulatory effects while leaving tissue undamaged. For example, in rat models of myocardial infarction and ischemia-reperfusion injury, low energy laser irradiation reduces infarct size and left ventricular dilation, and enhances angiogenesis in the myocardium. (Yaakobi et al., J. Appi. Physiol. 90, 2411-19 (2001)). Low level laser therapy has been described for treating pain, including headache and muscle pain, and inflammation. The use of low level laser therapy to accelerate bone remodeling and healing of fractures has also been described. (See, e.g., J. Tuner and L. Hode, LOW LEVEL LASER THERAPY, Stockholm:Prima Books, 113-16, 1999, which is herein incorporated by reference).

However, known low level laser therapy methods are circumscribed by setting only certain selected parameters within specified limits. For example, known methods are characterized by application of laser energy at a set wavelength using a laser source having a set power output. Specifically, known methods are generally typified by selecting a wavelength of the power source, setting the power output of the laser source at very low levels of 5 mW to 100 mW, setting low dosages of at most about 1-10 Joule/cm², and setting time periods of application of the laser energy at twenty seconds to minutes. However, other parameters can be varied in the use of low level laser therapy. In particular, known low-level laser therapy methods have not addressed the multiple other factors that may contribute to the efficacy of low level laser therapy.

Against this background, a high level of interest remains in finding new and improved therapeutic methods for overcoming organ transplant rejection, and for enhancing the survival time of a transplanted organ. A need also remains for therapeutic methods that prevent or retard organ transplant rejection while also reducing or avoiding administration of pharmaceutical agents having adverse side effects, particularly immunosuppressive agents.

### SUMMARY OF THE INVENTION

In a preferred embodiment there is provided a method for preventing or retarding rejection of a transplanted organ in a subject in need thereof. The method comprises delivering a rejection effective amount of light energy to the transplanted organ; the light energy having a wavelength in the visible to near-infrared wavelength range, wherein delivering the rejection effective amount of light energy comprises delivering a power density of light energy to the organ of at least about 0.01 mW/cm². The transplanted organ is preferably a solid organ, including but not limited to a heart, lung, kidney, liver, or pancreas.

In a preferred embodiment, delivering the power density of light energy to the organ comprises selecting a power and dosage of light energy sufficient to deliver a predetermined power density of light energy to the organ. Selecting a dosage and power of the light energy sufficient to deliver a predetermined power density of light energy to the site comprises, in one embodiment, selecting the dosage and power of the light sufficient for the light energy to traverse the distance and penetrate body tissue between the skin surface adjacent the transplanted organ and the organ.

In a preferred embodiment there is provided a method for preventing or retarding rejection of a transplanted organ in a subject in need thereof comprising administering to the subject an amount of an immunosuppressive agent and delivering to the transplanted organ an amount of light energy wherein the amount of the immunosuppressive agent and the amount of light energy together constitute a rejection effective amount, wherein delivering the amount of light energy comprises selecting a power density of the light energy. The transplanted organ is preferably a solid organ, including but not limited to a heart, lung, kidney, liver, or pancreas.

In a preferred embodiment there is provided a method for reducing the amount of an immunosuppressive agent or agents administered to a transplant subject to prevent or retard rejection of a transplanted organ. The method comprises delivering to the transplanted organ a rejection effective amount of light energy, the light energy having a wavelength in the visible to near-infrared wavelength range, wherein delivering the rejection effective amount of light energy comprises selecting a predetermined power density of light energy, the amount of light energy further sufficient to reduce the amount of immunosuppressive agent or agents required to prevent or retard rejection of the transplanted organ relative to the amount of immunosuppressive agent or agents required to prevent or retard rejection of the transplant organ without the use of light energy. The transplanted organ is preferably a solid organ, including but not limited to a heart, lung, kidney, liver, or pancreas.

Additional preferred embodiments of the foregoing methods may include one or more of the following: the selected power density is a power density selected from the range of about 0.01 mW/cm² to about 100 mW/cm²; the light energy has a wavelength of about 780 nm to about 840 nm; and the light is delivered in pulses at a frequency of about 1 Hz to about 1 kHz.

Preferred methods may further encompass selecting a dosage and power of the laser energy sufficient to deliver the predetermined power density of laser energy to the organ by selecting the dosage and power of the laser sufficient for the laser energy to penetrate any body tissue, for example a thickness of skin and other bodily tissue such as fat and muscle that is interposed between the transplanted organ and the skin surface adjacent the organ and/or sufficient for the laser energy to traverse the distance between the transplanted organ and the skin surface adjacent the organ.

### BRIEF DESCRIPTION OF THE DRAWINGS

FIG. 1 is a perspective view of a first embodiment of a light therapy device; and

FIG. 2 is a block diagram of a control circuit for the light therapy device, according to one embodiment of the invention.

### DETAILED DESCRIPTION OF THE PREFERRED EMBODIMENT

The low level laser therapy methods to prevent or retard rejection of a transplanted organ described herein are practiced and described using, for example, a low level laser therapy apparatus such as that shown and described in U.S. Pat. No. 6,214,035, U.S. Pat. No. 6,267,780, U.S. Pat. No. 6,273,905 and U.S. Pat. No. 6,290,714, which are all herein incorporated by reference together with the references contained therein.

A suitable apparatus for the methods to prevent or retard rejection of a transplanted organ is a low-level light apparatus including a handheld probe for delivering the light energy. The probe includes a laser source of light energy having a wavelength in the visible to near-infrared wavelength range, i.e. from about 630 nm to about 904 nm. In one embodiment, the probe includes a single laser diode that provides about 25 mW to about 500 mW of total power output, or multiple laser diodes that together are capable of providing at least about 25 mW to about 500 mW of total power output. In other embodiments, the power provided may be more or less than these stated values. The actual power output is preferably variable using a control unit electronically coupled to the probe, so that the power of the light energy emitted can be adjusted in accordance with required power density calculations as described below. In one embodiment, the diodes used are continuously emitting GaAIAs laser diodes having a wavelength of about 830 nm.

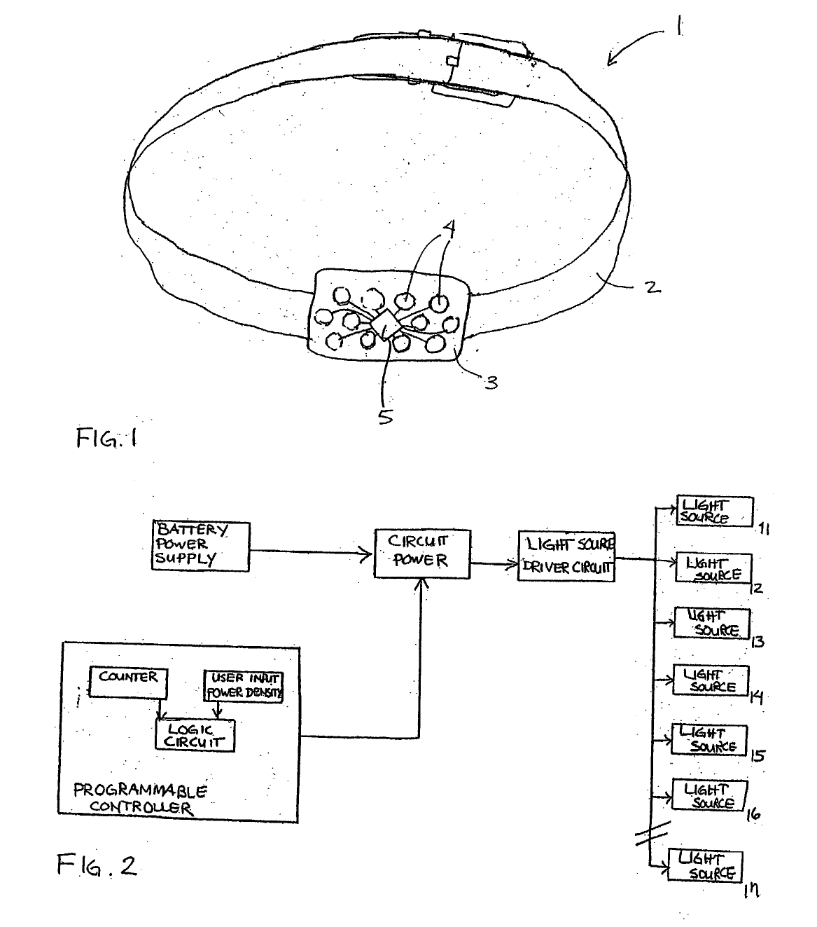

Another suitable light therapy apparatus is that illustrated in FIG. 1. This apparatus is especially preferred for methods in which the light energy is delivered through the skin. The illustrated device 1 includes a flexible strap 2 with a securing means, the strap adapted for securing the device over an area of the subject's body, one or more light energy sources 4 disposed on the strap 2 or on a plate or enlarged portion of the strap 3, capable of emitting light energy having a wavelength in the visible to near-infrared wavelength range, a power supply operatively coupled to the light source or sources, and a programmable controller 5 operatively coupled to the light source or sources and to the power supply. Based on the surprising discovery that control or selection of power density of light energy is an important factor in determining the efficacy of light energy therapy, the programmable controller is configured to select a predetermined surface power density of the light energy sufficient to deliver a predetermined subsurface power density to a body tissue to be treated beneath the skin surface of the area of the subject's body over which the device is secured.

The light energy source or sources are capable of emitting the light energy at a power sufficient to achieve the predetermined subsurface power density selected by the programmable controller. It is presently believed that tissue will be most effectively treated using subsurface power densities of light of at least about 0.01 mW/cm² and up to about 100 mW/cm², including about 0.05, 0.1, 0.5, 1, 5, 10, 15, 20, 30, 40, 50, 60, 70, 80, and 90 mW/cm². In one embodiment, subsurface power densities of about 0.01 mW/cm² to about 15 mW/cm² are used. To attain subsurface power densities within these stated ranges, taking into account attenuation of the energy as it travels through body tissue and fluids from the surface to the target tissue, surface power densities of from about 100 mW/cm² to about 500 mW/cm² will typically be required, but also possibly to a maximum of about 1000 mW/cm². To achieve such surface power densities, preferred light energy sources, or light energy sources in combination, are capable of emitting light energy having a total power output of at least about 25 mW to about 500 mW, including about 30, 50, 75, 100, 150, 200, 250, 300, and 400 mW, but may also be up to as high as about 1000 mW. It is believed that the subsurface power densities of at least about 0.01 mW/cm² and up to about 100 mW/cm² in terms of the power density of energy that reaches the subsurface tissue are especially effective at producing the desired biostimulative effects on tissue being treated.

The strap is preferably fabricated from an elastomeric material to which is secured any suitable securing means, such as mating Velcro strips, snaps, hooks, buttons, ties, or the like. Alternatively, the strap is a loop of elastomeric material sized appropriately to fit snugly over a particular body part, such as a particular arm or leg joint, or around the chest or hips. The precise configuration of the strap is subject only to the limitation that the strap is capable of maintaining the light energy sources in a select position relative to the particular area of the body or tissue being treated. In an alternative embodiment, a strap is not used and instead the light source or sources are incorporated into or attachable onto a piece of fabric which fits securely over the target body portion thereby holding the light source or sources in proximity to the patient's body for treatment. The fabric used is preferably a stretchable fabric or mesh comprising materials such as Lycra or nylon. The light source or sources are preferably removably attached to the fabric so that they may be placed in the position needed for treatment.

In the exemplary embodiment illustrated in FIG. 1, a light therapy device includes a flexible strap and securing means such as mating Velcro strips configured to secure the device around the body of the subject. The light source or sources are disposed on the strap, and in one embodiment are enclosed in a housing secured to the strap. Alternatively, the light source or sources are embedded in a layer of flexible plastic or fabric that is secured to the strap. In any case, the light sources are preferably secured to the strap so that when the strap is positioned around a body part of the patient, the light sources are positioned so that light energy emitted by the light sources is directed toward the skin surface over which the device is secured. Various strap configurations and spatial distributions of the light energy sources are contemplated so that the device can be adapted to treat different tissues in different areas of the body.

FIG. 2 is a block diagram of a control circuit according to one embodiment of the light therapy device. The programmable controller is configured to select a predetermined surface power density of the light energy sufficient to deliver a predetermined subsurface power density, preferably about 0.01 mW/cm² to about 100 mW/cm², including about 0.01 mW/cm² to about 15 mW/cm² and about 20 mW/cm² to about 50 mW/cm² to the target area. The actual total power output if the light energy sources is variable using the programmable controller so that the power of the light energy emitted can be adjusted in accordance with required surface power energy calculations as described below.

The methods described herein are based in part on the surprising finding that delivering low level light energy within a select range of power density (i.e. light intensity or power per unit area, in mW/cm²) appears to be an important factor for producing therapeutically beneficial effects with low level light energy as applied to a transplanted organ, to prevent or retard rejection of the transplanted organ. Without being bound by theory, it is believed that independently of the power and dosage of the light energy used, light energy delivered within the specified range of power densities provides a biostimulative effect on mitochondria to maintain cellular integrity and avoid tissue damage resulting from the triggering of biochemical cascades that cause ever-increasing tissue damage.

The term “organ” as used herein refers to a structure of bodily tissue in mammal such as a human being wherein the tissue structure as a whole is specialized to perform a particular body function. Organs that are transplanted within the meaning of the present methods include skin, cornea, heart, lung, kidney, liver and pancreas. Solid organs include the heart, lung, kidney, liver and pancreas.

The term “transplant”: as used herein refers to any organ or body tissue that has been transferred from its site of origin to a recipient site. Specifically in an allograft transplant procedure, the site of origin of the transplant is in a donor individual and the recipient site is in another, recipient individual.

The term “rejection” as used herein refers to the process or processes by which the immune response of an organ transplant recipient mounts a reaction against the transplanted organ sufficient to impair or destroy normal function of the organ. The immune system response can involve specific (antibody and T cell-dependent) or non-specific (phagocytic, complement-dependent, etc.) mechanisms, or both.

The term “rejection effective” as used herein refers to a characteristic of an amount of laser energy wherein the amount of laser energy achieves the goal of preventing, avoiding or retarding rejection of a transplanted organ, whether the process or processes underlying the rejection are specific or non-specific.

In preferred embodiments, treatment parameters include the following. Preferred power densities of light at the level of the target cells are at least about 0.01 mW/cm² and up to about 100 mW/cm², including about 0.05, 0.1, 0.5, 1, 5, 10, 15, 20, 30, 40, 50, 60, 70, 80, and 90 mW/cm². To attain subsurface power densities within this preferred range in in vivo methods, one must take into account attenuation of the energy as it travels through body tissue and fluids from the surface to the target tissue, such that surface power densities of from about 25 mW/cm² to about 500 mW/cm² will typically be used, but also possibly to a maximum of about 1000 mW/cm². To achieve desired power densities, preferred light energy sources, or light energy sources in combination, are capable of emitting light energy having a total power output of at least about 1 mW to about 500 mW, including about 5, 10, 15, 20, 30, 50, 75, 100, 150, 200, 250, 300, and 400 mW, but may also be up to as high as about 1000 mW or below 1 mW. Preferably the light energy used for treatment has a wavelength in the visible to near-infrared wavelength range, i.e., from about 630 to about 904 nm, preferably about 780 nm to about 840 nm, including about 790, 800, 810, 820, and 830 nm.

In preferred embodiments, the light source used in the light therapy is a coherent source (i.e. a laser), and/or the light is substantially monochromatic (i.e. one wavelength or a very narrow band of wavelengths).

In preferred embodiments, the treatment proceeds continuously for a period of about 30 seconds to about 2 hours, more preferably for a period of about 1 to 20 minutes. The treatment may be terminated after one treatment period, or the treatment may be repeated with preferably about 1 to 2 days passing between treatments. The length of treatment time and frequency of treatment periods can be varied as needed to achieve the desired result.

During the treatment, the light energy may be continuously provided, or it may be pulsed. If the light is pulsed, the pulses are preferably at least about 10 ns long, including about 100 ns, 1 ms, 10 ms, and 100 ms, and occur at a frequency of up to about 1 kHz, including about 1 Hz, 10 Hz, 50 Hz, 100 Hz, 250 Hz, 500 Hz, and 750 Hz.

Generally, light energy suitable for practicing the methods includes light energy in the visible to near-infrared wavelength range, i.e. wavelengths in the range of about 630 nm to about 904 nm. In an exemplary embodiment, the light energy has a wavelength of about 830 nm, as delivered with laser apparatus including GaAlAs diodes as the laser energy source.

Thus, in a preferred embodiment, methods directed toward preventing or retarding rejection of a transplanted organ in a subject in need thereof include delivering to the transplanted organ a rejection effective amount of light energy, the light energy having a wavelength in the visible to near-infrared wavelength range. In preferred embodiments, delivering the rejection effective amount of light energy comprises delivering a predetermined power density.

Delivering the predetermined power density of light energy to the transplanted organ involves determining or selecting the power density to be delivered, selecting a power and dosage of the light energy sufficient to deliver the predetermined power density of light energy to the organ, and applying the light energy directly to the transplanted organ or to a skin surface adjacent the transplanted organ. To deliver the predetermined power density of light energy to the transplanted organ, the location and orientation of the transplanted organ being treated should be considered, and an appropriate dosage and power of the light energy selected. The appropriate dosage and power of light energy are any combination of power and dosage sufficient to deliver the predetermined power density of light energy to the organ. In addition, when the delivery is performed transdermally, the dosage and power should be sufficient for the light energy to traverse the distance between the skin surface adjacent the transplanted organ and the organ including penetrating any body tissue that may be interposed between the skin surface adjacent the transplanted organ to which the light energy is applied, and the organ.

The methods are especially suitable for preventing, avoiding or retarding rejection of a transplanted solid organ including, but not limited to, a heart, a lung, a kidney, a liver, and a pancreas. However, other transplanted organs and tissues may also be beneficially treated using the methods.

It is understood that the specific power density selected for treating any specific transplanted organ in a given subject (transplant recipient) will be dependent upon a variety of factors including the age, gender, health, and weight of the subject, type of concurrent treatment if any (particularly immunosuppressive therapy), frequency of light energy treatments and, and the precise treatment goal and nature of the effect desired, such as whether the desired effect is to avoid acute or chronic rejection.

When the light therapy is delivered transdermally, a relatively greater surface power density of the light energy, as compared to the power density to be received at the transplanted organ, is calculated taking into account attenuation of the light energy as it travels from the skin surface where it is applied through various tissues including skin, muscle and fat tissue. Factors known to affect penetration and to be taken into account in the calculation of the required surface power density include skin pigmentation, and the location of the site being treated, particularly the depth of the site being treated relative to the skin surface. For example, to obtain a desired power density of about 10 mW/cm² at the site of injury or damage at a depth of 3 cm below the skin surface may require a surface power density of 400 mW/cm². The higher the level of skin pigmentation, the higher the required surface power density to deliver a predetermined power density of light energy to a subsurface site being treated

More specifically, to treat an organ transplant recipient to prevent or retard organ rejection, the light source is placed in contact with a region of skin adjacent the transplanted organ, for example a patch of skin on the lower abdomen adjacent a transplanted liver. The location and orientation of the transplanted organ can be determined as necessary by manual examination, or, if necessary by standard medical imaging techniques such as X-ray. The power density calculation takes into account factors including the location within the body of the organ transplant being treated, the extent and type of intervening body tissue such as fat and muscle between the skin surface, skin coloration, distance between the skin surface and the organ, etc. that affect penetration and thus power density that is actually received at the organ transplant. Power of the light source being used and the surface area treated are accordingly adjusted to obtain a surface power density sufficient to deliver the predetermined power density of light energy to the organ. The light energy source is then energized and the selected power density of light energy delivered to the organ.

In an exemplary embodiment, the light energy is applied to at least one treatment spot on the skin adjacent the transplanted organ, the spot having a diameter of about 1 cm. Thus, to fully treat a transplanted organ, which typically will have a surface area substantially larger than a spot having a diameter of about 1 cm, the light energy is applied sequentially to a series of multiple treatment spots having centers that are separated by at least about 1 cm. The series of treatment spots can be mapped out over the surface of the skin to aid in an orderly progression of light applications that systematically cover the surface area of the organ as it is being treated from any one approach. Some organs may be susceptible of treatment from more than one approach to the subject, i.e. treatment from the frontal and rear aspects of the subject, or from the frontal and side aspects. As the approach is changed, the surface power density needed to deliver the desired power density to the organ may change depending on whether and how the depth of the organ relative to the skin surface changes, and the nature and extent of intervening body tissue changes.

The power density selected for treating the patient is determined according to the judgment of a trained healthcare provider or light therapy technician and depends on a number of factors, including the specific wavelength of light selected, and clinical factors such as type of organ being treated, the current survival time of the organ, the clinical condition of the subject including the extent of immunosuppression, the location of the organ being treated, and the like. Similarly, it should be understood that the power density of light energy might be adjusted for combination with any other therapeutic agent or agents, especially pharmaceutical immunosuppressive agents to achieve the desired biological effect. The selected power density will again depend on a number of factors, including the specific light energy wavelength chosen, the individual additional immunosuppressive agent or agents chosen, and the clinical condition of the subject. Generally, applying the methods to routine transplant procedures entails applying the low level light energy to the transplanted organ any time after the transplant procedure is complete and preferably after the surgical site is closed. Specifically, the low level light energy is applied to a selected treatment spot or series of treatment spots adjacent the transplant site, at the power density determined in accordance with the judgment of a trained health care provider or light therapy technician, according to the method as described above. Thereafter, for as long as the graft survives, the light therapy can thereafter be applied on a regular basis such as daily or weekly. The light therapy can thus be used over a course of many months or even years, extending even over the lifetime of the transplant recipient to provide ongoing prevention or slowing of rejection for as long as the transplant survives.

The light therapy methods as described herein can also be advantageously used in combination with an immunosuppressive agent or agents. Use of the light therapy methods as described herein reduces the amount of immunosuppressive agent or agents needed to prevent, avoid or retard graft rejection. For example, immunosuppressive agents used in accordance with the light therapy methods include cyclosporine and tacrolimus, adrenocortical steroids including prednisone and prednisalone, cytotoxic drugs including azathioprine, mycophenolate mofetil, cyclophosphamide, methotrexate, chlorambucil, vincristine, vinblastine and dactinomycin, and antibody reagents including antithymocyte globulin, muromonab-CD3 monoclonal antibody and Rho(D) immune globulin. The immunosuppressive agents are generally administered orally, intravenously or intramuscularly, but may alternatively be administered using other known routes of administration including subcutaneous injection, sublingual, and intraperitoneal injection.

For example, cyclosporine is orally or intravenously administered once daily starting 4 to 24 hours post transplantation at a dose of about 15 mg/kg. The dose is continued for 1 to 2 weeks post-operatively. Thereafter the dose is reduced each week until a maintenance dose of about 3 mg/kg to about 10 mg/kg is reached. In a preferred embodiment, light therapy is also administered once daily starting about 4 to 24 hours post transplantation. Using concurrent light therapy, the maintenance dose of cyclosporine should be reduced relative to the amount of cyclosporine that would otherwise be required to achieve the desired therapeutic without adjunct light therapy. Reducing the maintenance dose of cyclosporine in particular as described using adjunct light therapy is particularly desirable for kidney transplant recipients because of the renal toxicity of cyclosporine.

The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention.

What is claimed is:

1. A method for preventing or retarding rejection of a transplanted organ in a subject in need thereof, said method comprising delivering a rejection effective amount of light energy to the transplanted organ; the light energy having a wavelength in the visible to near-infrared wavelength range, wherein delivering the rejection effective amount of light energy comprises delivering a power density of light energy to the organ of at least about 0.01 mW/cm².

2. A method in accordance with claim 1 wherein the selected power density is a power density selected from the range of about 0.01 mW/cm² to about 100 mW/cm².

3. A method in accordance with claim 1, wherein the light energy has a wavelength of about 780 nm to about 840 nm.

4. A method in accordance with claim 1, wherein the light is delivered in pulses at a frequency of about 1 Hz to about 1 kHz.

5. A method in accordance with claim 1 wherein delivering the power density of light energy to the organ comprises selecting a power and dosage of light energy sufficient to deliver a predetermined power density of light energy to the organ.

6. A method in accordance with claim 5 further comprising applying the light energy to a skin surface adjacent the transplanted organ.

7. A method in accordance with claim 6 wherein selecting a dosage and power of the light energy sufficient to deliver a predetermined power density of light energy to the site comprises selecting the dosage and power of the light sufficient for the light energy to traverse the distance and penetrate body tissue between the skin surface adjacent the transplanted organ and the organ.

8. A method in accordance with claim 1 wherein the transplanted organ is a solid organ.

9. A method in accordance with claim 8 wherein the solid organ is selected from the group consisting of a heart, a lung, a kidney, a liver, and a pancreas.

10. A method for preventing or retarding rejection of a transplanted organ in a subject in need thereof comprising administering to the subject an amount of an immunosuppressive agent and delivering to the transplanted organ an amount of light energy wherein the amount of the immunosuppressive agent and the amount of light energy together constitute a rejection effective amount, wherein delivering the amount of light energy comprises selecting a power density of the light energy.

11. A method in accordance with claim 10 wherein the selected power density is a power density selected from the range of about 0.01 mW/cm² to about 100 mW/cm².

12. A method in accordance with claim 10 wherein the light energy has a wavelength of about 630 nm to about 904 nm.

13. A method in accordance with claim 10, wherein the light energy has a wavelength of about 780 nm to about 840 nm.

14. A method in accordance with claim 10, wherein the light is delivered in pulses at a frequency of about 1 Hz to about 1 kHz.

15. A method in accordance with claim 10, wherein the transplanted organ is a solid organ.

16. A method in accordance with claim 15 wherein the solid organ is selected from the group consisting of a heart, a lung, a kidney, a liver, and a pancreas.

17. A method for reducing the amount of an immunosuppressive agent or agents administered to a transplant subject to prevent or retard rejection of a transplanted organ, said method comprising delivering to the transplanted organ a rejection effective amount of light energy, the light energy having a wavelength in the visible to near-infrared wavelength range, wherein delivering the rejection effective amount of light energy comprises selecting a predetermined power density of light energy, the amount of light energy further sufficient to reduce the amount of immunosuppressive agent or agents required to prevent or retard rejection of the transplanted organ relative to the amount of immunosuppressive agent or agents required to prevent or retard rejection of the transplant organ without the use of light energy.

18. A method in accordance with claim 17 wherein the selected power density is a power density selected from the range of about 0.01 mW/cm² to about 100 mW/cm².

19. A method in accordance with claim 17, wherein the light energy has a wavelength of about 780 nm to about 840 nm.

20. A method in accordance with claim 17, wherein the light is delivered in pulses at a frequency of about 1 Hz to about 1 kHz.

21. A method in accordance with claim 17 wherein the transplanted organ is a solid organ.

22. A method in accordance with claim 21 wherein the solid organ is selected from the group consisting of a heart, a lung, a kidney, a liver, and a pancreas.
